(19) United States
(12) Patent Application Publication
(10) Pub. No.: US 2011/0180449 A1
(43) Pub. Date: Jul. 28, 2011

(54) HAIR CARE COMPOSITION

(76) Inventor: Patti D. Rubin, Aventura, FL (US)

(21) Appl. No.: 12/845,282

(22) Filed: Jul. 28, 2010

Related U.S. Application Data
(63) Continuation-in-part of application No. 11/840,014, filed on Aug. 16, 2007, now abandoned.
(60) Provisional application No. 60/822,619, filed on Aug. 16, 2006.

(51) Int. Cl. A61Q 5/00; A61K 8/11; A61K 8/65; A61K 8/73; A61K 8/81; A61Q 5/12; A61Q 5/06; A61Q 5/02; B65B 29/00; A45D 34/00

(52) U.S. Cl. 206/581; 424/401; 424/70.6; 424/70.14; 424/70.13; 424/70.16; 424/70.17; 424/70.11; 510/119; 53/428; 206/37

(57) ABSTRACT

A hair care solution configured to reduce volume and size of the product includes a hydrophilic polymer, and a hair care ingredient encapsulated in the water-absorbent polymer. Accordingly, the encapsulated hair care ingredient is released for use when the hydrophilic polymer is activated with water.

### RELATED APPLICATIONS

The present application is a continuation-in-part of U.S. patent application Ser. No. 11/840,014 filed Aug. 16, 2007, titled “Hair Care Composition,” which claims the benefit under 35 U.S.C. §119(e) of U.S. Provisional Patent Application No. 60/822,619 filed Aug. 16, 2006 titled “Hair Care Composition,” the disclosures of which applications are hereby incorporated by reference in their entireties.

### BACKGROUND

Hair Care products, such as shampoo and conditioner are well-known. Often, it is desirable to transport hair care products. For example, it may be desirable to transport hair care products or other items when traveling or heading to the outdoors for extended periods. Currently, hair care products are stored and carried in bottles or other similar containers. When packed, these containers are frequently in close contact with the other articles that are packed to be transported. Occasionally, these containers leak, such that the hair care products are spilled onto the other packed articles, resulting in inconvenience or even damage to the other packed articles. Particularly, when traveling by air, the change in pressures as the airplane ascends and descends thousands of feet can cause the traditionally liquid hair care products to leak from their containers. Additionally, the traditionally liquid based hair care products occupy relatively large volumes.

### BRIEF DESCRIPTION OF THE DRAWINGS

The accompanying drawings illustrate various embodiments of the present system and method and are a part of the specification. The illustrated embodiments are merely examples of the present system and method and do not limit the scope thereof.

FIG. 1 is a simple block diagram illustrating a method of forming a hair care composition, according to one exemplary embodiment.

FIG. 2 is a simple block diagram depicting a method of forming a hair care composition, according to one exemplary embodiment.

FIG. 3 is a simple block diagram illustrating a method of forming a hair care composition, according to one exemplary embodiment.

FIG. 4 is a simple block diagram illustrating a method of forming a hair care composition, according to another exemplary embodiment.

FIG. 5 is a simple schematic illustrating a system for compressing and shaping a hair care composition, according to one exemplary embodiment.

FIG. 6 is a simple schematic illustrating a system for compressing and shaping a hair care composition, according to another exemplary embodiment.

FIG. 7 is a perspective view of packaging including a plurality of hair care composition strips, according to one exemplary embodiment.

FIGS. 8A and 8B are partial cross-sectional views of a sealed volume containing hair care composition according to an embodiment of the present invention.

Throughout the drawings, identical reference numbers designate similar, but not necessarily identical, elements.

### DETAILED DESCRIPTION

The presently described exemplary systems and methods provide hair care compositions and methods for forming hair care compositions. One exemplary hair care composition includes a swellable agent, such as hydrophilic polymerics combined with dehydrated hair care products. Consequently, the present exemplary hair care composition occupies minimal space, is resistant to changes in pressure, and may be readily rehydrated and used when desired. Accordingly, the present exemplary hair care composition essentially eliminates the possibility that a hair care composition will be spilled when packed or during travel. Further such a configuration may reduce the volume occupied by the hair care composition, thereby making packing the hair care composition more convenient. Further details of exemplary hair care compositions, as well as exemplary formation methods will be provided below with reference to the exemplary figures. Moreover, exemplary embodiments provide packaging of hair care compositions that enable the access of individual doses or treatments of a hair compositions while preserving other doses or treatments of hair care compositions.

In the following description, for purposes of explanation, numerous specific details are set forth in order to provide a thorough understanding of the present systems and methods. It will be apparent, however, to one skilled in the art that the present apparatus, systems and methods may be practiced without these specific details. Reference in the specification to “an embodiment,” “an example” or similar language means that a particular feature, structure, or characteristic described in connection with the embodiment or example is included in at least that one embodiment, but not necessarily in other embodiments. The various instances of the phrase “in one embodiment” or similar phrases in various places in the specification are not necessarily all referring to the same embodiment.

According to one exemplary embodiment, the present hair care composition includes a polymer capsule and a hair care ingredient. Exemplary materials that may be used as the hair care composition and the polymer capsule will be provided below.

According to the present exemplary system and method, one or more hair care ingredients is encapsulated in a polymer capsule, to be released when activated with water or some other hydrating medium. According to this exemplary embodiment, any number of polymers may be used to encapsulate the hair care composition products. Exemplary polymers suitable for use in the present exemplary hair care composition include any suitable polymer including, but not limited to, a wide variety of anionic, cationic, and nonionic materials. Suitable polymers include, but are in no way limited to, acrylic polymers such as acrylamides, acrylates, and co-polymers thereof; poly(alkylene oxides) such as poly(ethylene oxide); cross-linked polyethylene oxide co-polyurethane hydrogel; polyvinyl alcohols; ethylene maleic anhydride copolymer; polyvinylethers; polyacrylic acids; polyvinylpyrrolidones; polyvinylmorpholines; polyamines; polyethyleneimines; polyquaternary ammoniums; saponified copolymers of vinyl acetate-acrylic acid ester; and hydrolyzed copolymers of acrylonitrile or acrylamide, or crosslinked polymers of these hydrolyzed copolymers; partially-neutralized crosslinked poly(acrylic acid); natural based polysaccharide polymers such as methyl celluloses, carboxymethyl celluloses, carboxymethyl starches, hydroxypropyl celluloses, algins, alginates, carrageenans, acrylic grafted starches, acrylic grafted celluloses, chitin, chitosan, starch-acrylonitrile, neutralized graft polymers of starch-acrylic acid; and synthetic polypeptides such as polyaspartic acid, polyglutamic acid, polyasparagins, polyglutamines, polylysines, and polyarginines; as well as the salts, copolymers, cross-linked derivatives and mixtures of any of the foregoing polymers. The polymers may be hydrophilic (water soluble) or hydrophobic (water insoluble) according to various embodiments. Cross-linking hydrophilic polymers can increase the insolubility of the cross-linked compound. Additionally, cross-linked compounds have the capability of absorbing great amounts of water.

In one exemplary embodiment the polymer capsule of the present exemplary hair care composition is made from a potassium- or sodium-based polymer, such as a synthetic polyacrylate/polyacrylamide copolymer. Like many absorbent polymers, synthetic polyacrylate/polyacrylamide copolymer can absorb many hundred times its weight in water, thereby allowing for a reduction in size of the cosmetic as well as minimizing or reducing the use of preservatives associated with the cosmetic. In another exemplary embodiment, the absorbent polymer capsule is acrylamide/potassium acrylate copolymer.

According to one exemplary embodiment, the polymer capsule component of the present exemplary hair care composition carries a hair care ingredient that can be released upon hydration to form a fully-hydrated and/or full-sized hair care composition that is ready for application.

As mentioned, the present exemplary hair care composition includes a hair care ingredient that is released when the composition is hydrated or otherwise activated with water. Any number of complete hair care products may be included as a hair care composition of the present exemplary hair care composition including, but in no way limited to, shampoo, conditioner, anti curl lotion, anti humectant pomade, color enhancing conditioner, color glaze, color mousse, color treated hair conditioner, colored hair shampoo, corrective styling mousse, cover gray, curl defining gel, curl defining shampoo, dandruff shampoo, defining cream, detanglers, ethnic conditioner, ethnic relaxer, ethnic shampoo, foam mask, foaming pomade, foaming styler, gel, hair gloss, hair loss shampoo, hydrating masque, straighteners, keratin treatment, tonic, molding cream, non-permanent hair color, pre-shampoo treatment, protecting spray, regrowth treatment, relaxing serum, restructuring serum, root lift, root pump, sculpting gel, shine pomade, silk therapy, smoothing cream, smoothing mask, smoothing serum, straightening balm, strengtheners, thickening lotion, vitamins, volume booster, volumizing conditioner, and/or volumizing gel.

In the case of color ingredients, a solvent, such as peroxide, may be incorporated with the use of the hair care composition. For example, peroxide may be added during rehydration of the hair care composition, or the peroxide or other solvent may be incorporated with the color so as to activate when exposed to water or some other solvent.

The present exemplary hair care composition can include any number of natural or organic ingredients including, but in no way limited to, aloe derivatives, aloe barbadensis gel, alpha lipoic acid, aleurites muluccana seed oil, ascorbyl palmitate, apricot kernel oil, aqua, basil, behentrimonium methosulfate, calendula extract, chamomile extract, castor oil, carnauba, cetyl alcohol, citronellol, coumarin, citric acid, sodium sweetalmondamphoacetate, geranium oil, hydrolyzed wheat protein, jojoba oil, kelp, kelp extract, lanolin, macadamia oil, palmitic acid, panthenol (pro Vitamin B5), rosemary extract, safflower oil, shea butter, sodium ascorbyl phosphate, sorbitol, stearic acid, sucrose stearate, teatree extract, and/or tocopheryl acetate.

Additionally, the present exemplary hair care composition can include Acetamide MEA, Alcohol, Algae Extract, Algal Polysaccharides, Allantoin, AMP, Ammonium Lauryl Sulfate, Amphoteric Surfactants, Annatto Extract-annionic Surfactants, Beet Extract-Benzophenone, Beta Carotene, Biotin, Boric Acid, Butylene Glycol, Caramel, Carbomer 940, Carrageenan, Cationic Surfactants, Ceteareth-5, Cetearyl Alcohol, Ceteth-2, Ceteth-20, Cetrimonium Bromide, Cetrimonium Chloride, Cetyl Alcohol, Cetyldimonium Chloride, Chloroxylenol, Cocamide DEA, Cocamide MEA, Cocamidopropyl Betaine, Coco Betaine, Cyclomethicone, DEA Oleth-3 Phosphate, DEA Oleth-10 Phosphate Diazolidinyl, Dicetyldimonium Chloride, Dimethicone, Dimethicone Copolyol, Dimethyl Lauramine Isostearate, Dimethyl Stearamine, EDTA Ethyl Ester PVM/MA Copolymer, Essential Oils, Glyceryl Monstearate, Glyceryl Stearate, Glycolic Acid, Glycol Stearate, Grapeskin Extract, Green Tea Extract, Guar Hydroxypropyltrimonium Chloride, Hyaluronic Acid, Hydrolyzed Human Hair Keratin Protein, Hydroxyethel Cellulose, Hydroxypropyl Methylcellulose, Isobutane, Isopropanol, Isopropyl Alcohol (Isopropyl Palmitate, Lactamide MEA Lactic Acid, Laureth-3, Lecithin, Lineolamido Propyl Ethydimonium Ethosulfate, Magnesium Citrate, Methacryloyl Ethyl Betaine Methylchloroisthiazolinone, Methylisothiazolinone, Methyl Paraben, Myristalkonium Chloride, Niacinamide, Nonionic Surfactants, Nonoxynol 12-O, Cresol, Octylacrylamide Acrylate Butylaminoethyl Methacrylate Copolymer, Octylacrylamide Butylaminoethyl Methacrylate Copolymer, Octyl Methoxycinnamate, Oleth 20, Orange Peel Extract, Palm Kernelamide DEA and MEA Panthenol, PEG, Pentacrythritol Tetra Caprate/Caprylate, Phenyl Trimethicone, Polyquaternium 11, Polysorbate 20, Polysorbate 80, Potassium Sorbate, PPG 2 Isodeceth 12, Pristane, Propane, Propyl Paraben, Propylene Glycol Dicocoate, Pyroxidine HCL, Quaternium 15, Salicylic Acid, SD 40 Alcohol, SD Alcohol 40B, Shea Butter, Sodium Cetyl Sulfate, Sodium Hydroxymethylglycinate, Sodium Laureth Sulfate, Sodium Myristoyl Sarcosinate, Sodium PCA, Sodium Thiosulfate, Sorbitol, Stearalkonium Chloride, Stearamidopropyl Dimethyamine, Steareth 21, Stearic Acid, Stearyl Alcohol, Surfactant, TEA Laureth Sulfate, TEA Lauryl Sulfate, Tetrasodium EDTA, Triethanolamine (TEA) and Xanthan Gum. Further, the present exemplary hair care composition can include a scented polymer to provide a desired scent to the hair care composition.

As used in the present exemplary specification, and in the appended claims, the above-mentioned products, ingredients, and their equivalents, both individually and in any feasible combination, shall be referred to as “hair care ingredients.” According to the present exemplary system and method, the hair care ingredients may be rehydrated by the rehydration or other activation of the hydrophilic polymer capsules.

The concentration of the hair care ingredient can be 100% concentrated, or alternatively, the hair care ingredient may be diluted with water or an organic solvent. According to one exemplary embodiment, the hair care ingredient typically ranges from about 0.01% to about 99.9% by weight of the present exemplary hair care composition. According to one exemplary embodiment, the hair care ingredient ranges from about 80.00% to about 99.9% by weight of the present exemplary hair care composition. Essentially any range of hair care ingredient may be included, depending on the capabilities of the polymer.

Generally, the hair care composition includes one or more polymer capsules in which the hair care ingredient inside the capsule is a solid, liquid, or gas. According to one exemplary embodiment, the present exemplary hair care composition is prepared by microencapsulation of the hair care ingredient. Appropriate microencapsulation processes include both physical and chemical techniques, as will be described in further detail below.

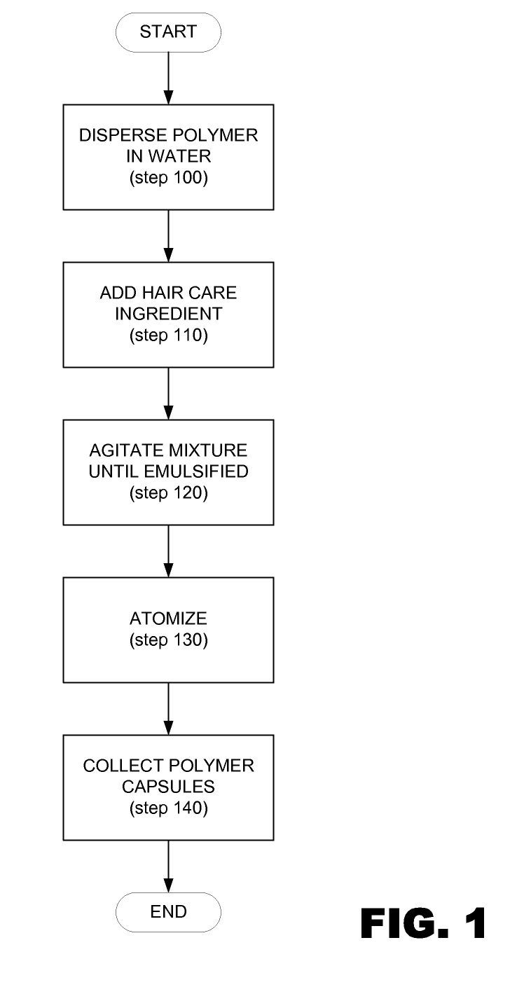

According to one exemplary embodiment, physical methods use commercially available equipment to create and stabilize the capsules. Particularly, in one exemplary embodiment, the microencapsulation is performed by a spray-drying method, as illustrated in FIG. 1. Generally, the spray drying process begins by creating an emulsion of the capsule polymer and hair care ingredient. In one embodiment, as shown in FIG. 1, the emulsion is made by dispersing or dissolving the capsule polymers in a liquid solution such as water (step 100). Once the polymer has been dispersed or dissolved in a solution, the hair care ingredient is slowly added (step 110) and the mixture is rapidly agitated until such time as emulsification is complete (step 120).

Once an emulsion has been created, the liquid emulsion is atomized (step 130) into a heated air stream supplied to a drying chamber. The spray-drying process uses a two-nozzle (internal or external mix) assembly, allowing the heated air from an annular geometry to atomize and implode the issuing liquid stream to form fine polymer capsules carrying the microencapsulated hair care ingredient in a dispersed state, according to one exemplary embodiment. These atomized particles assume a somewhat spherical shape as they fall through the gaseous medium, and the hair care ingredient is encased in the aqueous phase. With high, particle-specific surface areas, heat from the drying chamber flash-evaporates the solvent or aqueous component, rendering the polymer capsules cyclone-collected into a holding chamber (step 140).

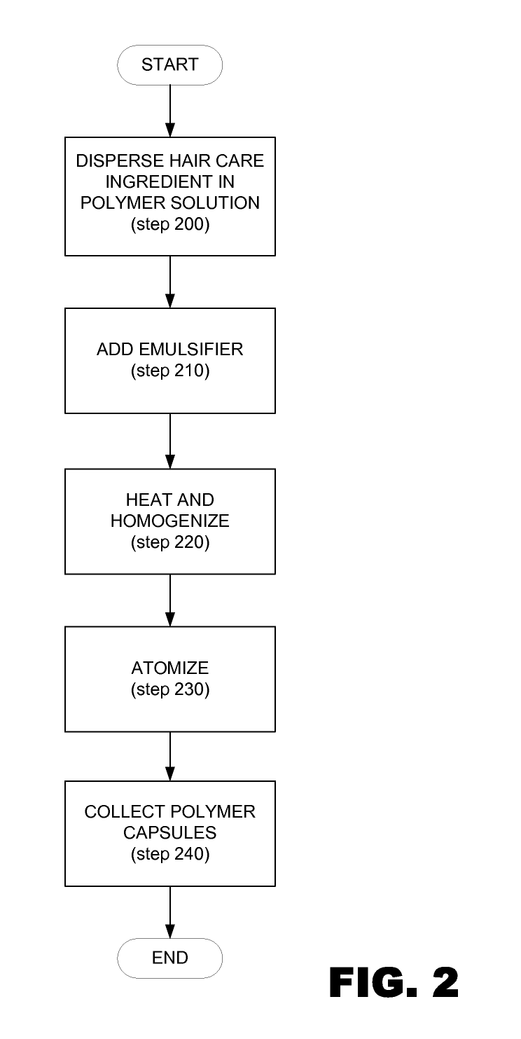

In another exemplary embodiment, as shown in FIG. 2, the emulsion is made by dispersing at least one hair care ingredient in a polymer solution containing the polymer capsule material to create a dispersion (step 200). An emulsifier is then added to the dispersion (step 210) and the dispersion is then heated and homogenized (step 220). This homogenization creates an oil-in-water type of emulsion. Additionally, a cross-linking agent may be added to the emulsion(s) to enhance the water absorbing ability of the resulting capsule. Once the emulsion has been created, the liquid emulsion is atomized (step 230) to form polymer capsules. The polymer capsules are then collected (step 240) for use in the hair care composition.

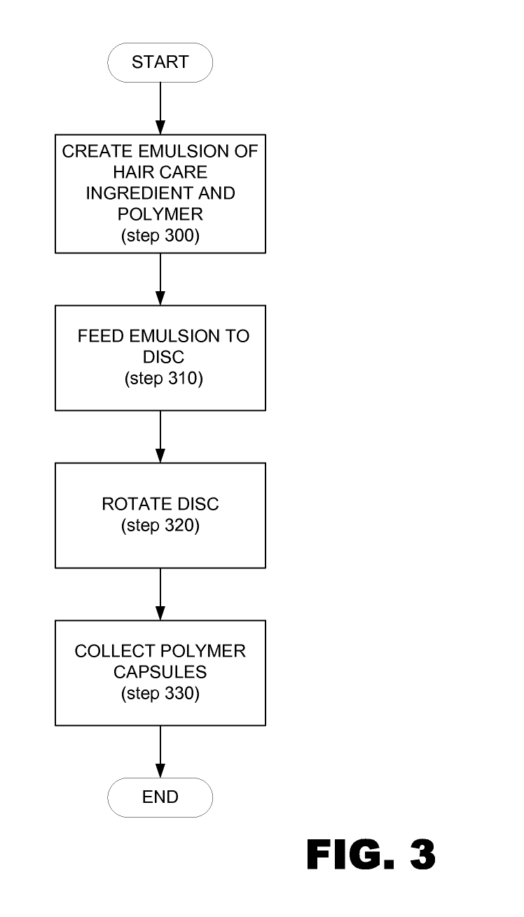

In yet another exemplary embodiment, illustrated in FIG. 3, the microencapsulation technique used to form the polymer capsules includes a spinning disc process. According to the exemplary method illustrated in FIG. 3, an emulsion or suspension containing the hair care ingredient is first prepared (step 300) with a solution or melt of the coating material, similar to the spray-drying process illustrated in FIGS. 1 and 2. Once prepared, the emulsion or suspension is fed to a disc surface (step 310) where it forms a thin wetted layer. Once the emulsion or suspension is fed to the disk surface, the disk is caused to rotate (step 320). During rotation of the disk, the thin layer of emulsion or suspension breaks up into airborne droplets due to surface tension forces that induce thermodynamic instabilities, resulting in spherical capsules that are then collected (step 330). According to one exemplary embodiment, the spinning disk process illustrated in FIG. 3 allows the use of a higher viscosity shell material and allows higher loading of the hair care ingredient in the shell. A higher viscosity shell material may be used because the emulsion or suspension is not extruded through an orifice. Additionally, the spinning disk process also offers a broad range of particle sizes that may be formed with controlled distribution by varying the conditions of the rotating disk.

In yet another exemplary embodiment, the microencapsulation technique involves coextrusion encapsulation methods. According to this exemplary embodiment, the polymer capsules may be formed using stationary nozzle coextrusion, centrifugal coextrusion, or submerged nozzle coextrusion. All these processes involve concentric nozzles that pump the hair care ingredients through an inner nozzle while the shell formulation is pumped through the annulus, allowing true “core-shell” morphologies. As the liquid stream exits the nozzle, local disturbances, such as induced vibration or gravitational, centrifugal, or drag force, control particle size. Typical microcapsules produced by coextrusion may range from approximately 100 micrometers to 6 mm.

The microencapsulation processes may further include chemical processes such as phase separation, gelation, and simple or complex coacervation. In one exemplary embodiment, the microencapsulation technique involves phase separation in which the hair care ingredient is emulsified in a polymer solution and an antisolvent is subsequently added to induce the precipitation of the polymer around the hair care ingredient. In yet another exemplary embodiment coacervation is used. According to this exemplary technique, microcapsule shells are formed by ionic interaction between two ionic polymers, typically a polyanion and a polycation. In another embodiment, gelation is used as the microencapsulation method and involves using a technique such as cooling, crosslinking, or a chemical reaction to form gelled microspheres or microcapsules.

It should be understood that the present system and methods may also include the use of any other microencapsulation technique known to those of skill in the art. Examples of these methods include, but are not limited to, vibrating nozzle, pan coating, fluid bed, spray coating, interfacial polymerization, solvent evaporation, in situ polymerization, liposome, sol-gel methods, nanoencapsulation, and others.

According to the present exemplary system and method, once the microcapsules are generated, they may then go through any number of processes to prepare the present exemplary hair care composition for presentation to a user. According to one exemplary embodiment, the hair care composition can be presented to the user in a microencapsulated form. Alternatively, according to one exemplary embodiment, the hair care composition may be compressed or otherwise processed such that the hair care composition is formed into individual units, such as thin strips, tabs, capsules, or any other desired units of a desired size. According to one exemplary embodiment, the present exemplary hair care composition is formed by joining multiple microcapsules with a hydrophilic binder into a strip form. These strips may then be dispensed to a user in a rolled tape dispenser type configuration. Alternatively, the strips may be cut into individual single dose sections. These individual units may then be hydrated, such as by adding water while the user is in a bathing environment, such as a shower, bath, or stream. Such a configuration provides for convenient use of the hair care composition by providing a proper amount of the composition for a single application. Further, such a configuration reduces the volume occupied by the composition, thereby making packing more convenient and further reduces the possibility that the composition will contaminate or otherwise damage other items packed therewith.

Additionally, according to one exemplary embodiment, multiple microcapsules containing different hair care ingredients may be mixed according to a desired ratio and joined with a hydrophilic binder. Specifically, by encapsulating the hair care ingredient, infinite combinations can be produced without re-designing the entire manufacturing line. According to this exemplary embodiment, different hair care properties, scents, and/or formulations can be provided by varying the microcapsule formulation prior to binding the various microcapsules. Alternatively, various microencapsulated hair care ingredients can be provided to the user, allowing the user to mix and match ingredients as they desire.

The polymer encapsulates the hair care ingredient, thereby protecting it until the release of the hair care ingredient is induced. Many different mechanisms may trigger the release of the hair care ingredient. In one exemplary embodiment, the polymer is configured to release the hair care ingredient through micropores created in the surface of the polymer when the addition of water causes the polymer to swell to a larger size. Release of the hair care ingredient in the presence of water may be further facilitated by using hydrophobic hair care ingredients that are repelled by absorbed water.

In an alternative exemplary embodiment, the polymer capsule is configured to release the hair care ingredient by dissolving in the presence of water. Other release mechanisms that may be used to systematically release the hair care ingredient include, but are in no way limited to, mechanical polymer rupture, thermal release, permeation, dissolution, delayed and targeted release, pH and osmotic release, photolytic release, biodegradation, and other release methods known to those of skill in the art. According to any number of the mentioned release mechanisms, a fully constituted hair care product can be produced via rehydration.

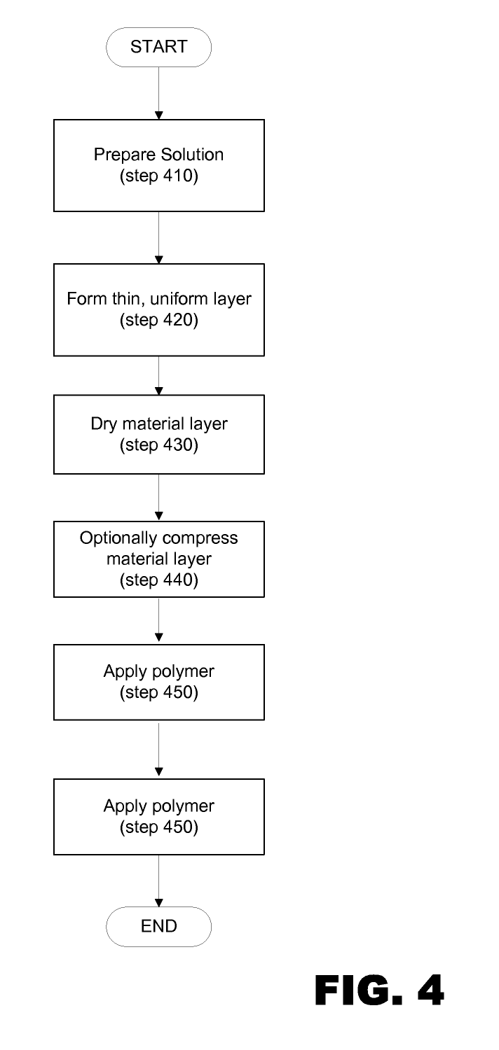

With reference to FIG. 4 another exemplary embodiment of preparing a hair care composition is illustrated. A solution including the desired hair care ingredients (e.g., shampoo, conditioner, or color ingredients) is prepared (step 410). The solution may then be spread into a relatively thin and generally uniform layer of material (step 420) such as by spinning within a centrifugal drum or spreading over a surface of a mold cavity. After the material layer exhibits an desired level of dehydration, such that it may be handled for collecting and further processing, the material may be removed from the centrifugal drum, mold or other structure and then further dried (step 430). The drying or dehydration of the material layer may be accomplished by a variety of processes including, for example, exposing the material layer to ambient conditions for a specified period of time, or by exposing the material layer to a heat source, such as a heat tunnel, to expedite the drying of the material layer. The material layer may optionally be compressed (step 440) such as by application of pressure by a compaction roller, by vacuum bagging, or by some other appropriate process.

In one embodiment, a polymer material may be applied to the dried material layer (step 450) to maintain the dehydrated composition in a dried state until exposed to water or an appropriate solvent so as to rehydrate the hair care composition such as has been described above with respect to other exemplary embodiments. The application of a polymer material may include the use of a scented material to provide the resulting hair care composition with a desired scent. Either prior to performing step 450, or after performing step 450, the material layer may be cut into strips of desired dimensions. The strips may then be packaged (step 460) in preparation for shipping, distribution and storage. The hair care composition, in the form of individual strips, may be packaged individually (i.e., one strip being placed in a sealed volume) so that when a user accesses a strip (e.g., to shampoo, condition or color their hair), the do not expose any other strips to moisture.

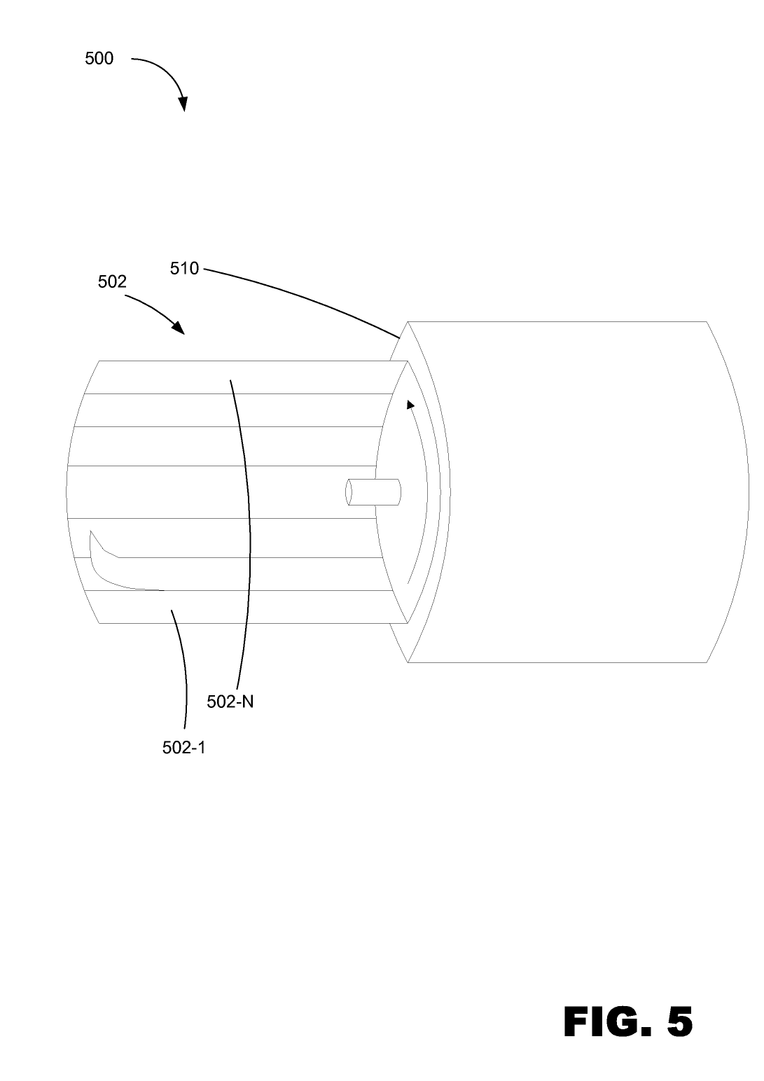

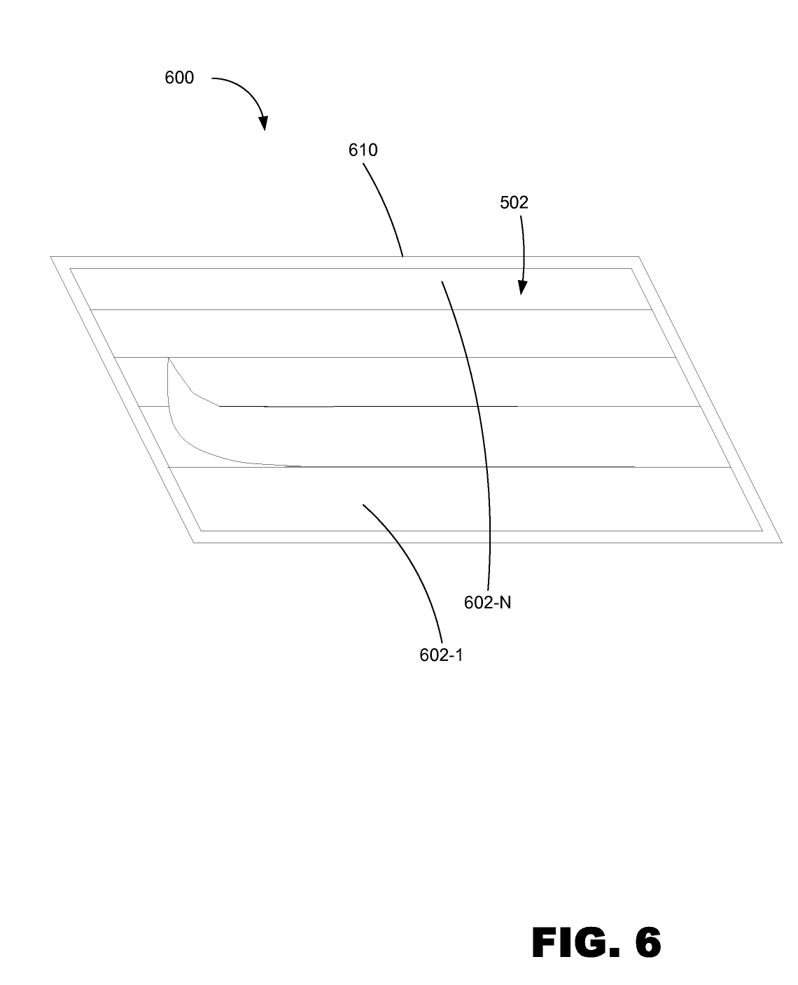

FIGS. 5 and 6 illustrate two exemplary embodiments for forming a material layer as set forth with respect to step 420. With respect to FIG. 5, the prepared solution may be placed in a centrifugal drum 500 to form a thin material layer 502. In this exemplary embodiment the drum 510 rotates at speeds sufficient to force the solution (or other prepared materials) radially outward against the drum wall to create a substantially uniform film. In one embodiment, the resulting material layer 502 may be cut into smaller portions (502-1 through 502-N, collectively) and peeled off of the drum wall. These smaller portions (e.g., 520-1) may then be further dried and processed as previously described.

Referring briefly to FIG. 6, another exemplary embodiment is shown of preparing a material layer as set forth in step 420. The process may include disposing a solution into a mold pan 610 and letting gravity distribute the solution into a substantially uniform layer of material 602. As noted above, the material may be compressed if desired. Once dried, portions (e.g., 602-1) may be may be formed into a final shape (e.g., cut into strips) and packaged such as previously described. These and other systems or methods known to those of skill in the art may also be used to compress, shape, or dry hair care microcapsules.

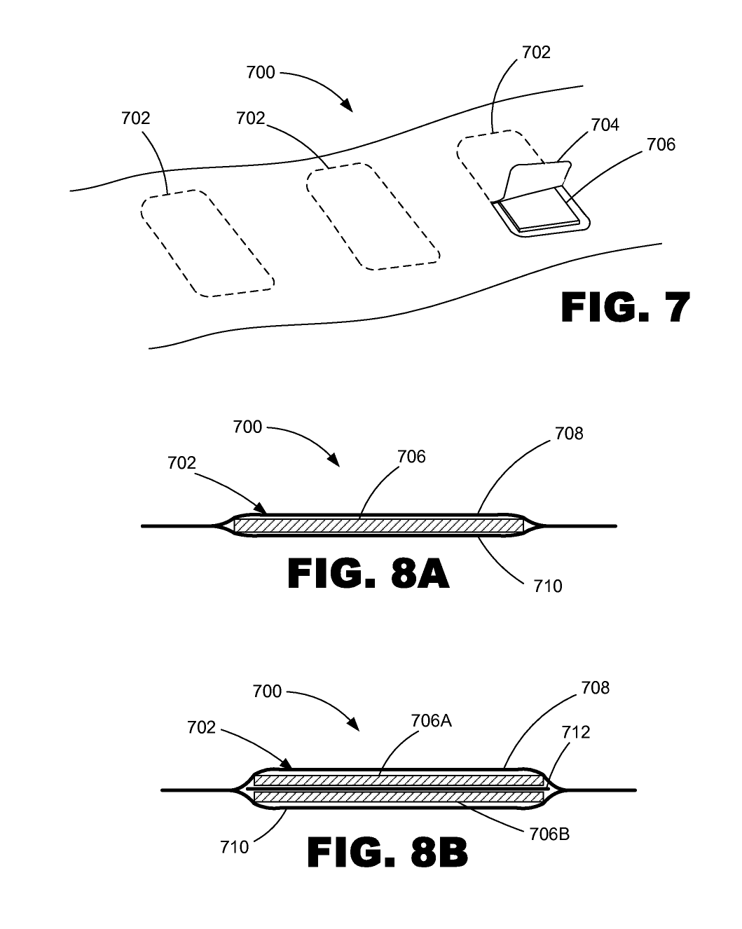

FIG. 7 illustrates an exemplary embodiment of packaging 700 containing a hair care composition, in the form of a plurality of discrete strips, that are disposed in individual fluid tight volumes 702. Each volume 702 may be formed so that a portion 704 of the packaging material defining the volume 702 may be peeled back or retracted to expose the discrete strip 706 (or other form) of hair care composition disposed within the volume 702. The packaging material may include any of a variety of different materials and, in one embodiment, may be formed of a biodegradable material. As previously noted, the packaging of individual discrete components (e.g., strips 706) enables a user to access a desired volume or dose of hair care composition without prematurely exposing other volumes of doses (e.g., strips 706) to moisture and inadvertently rehydrating them.

Referring to FIG. 8A, a partial cross-sectional view is shown of a hair care composition strip 706 disposed in a sealed volume 702 of the packaging shown in FIG. 7 according to one exemplary embodiment. The strip 706 is disposed within the volume 702 defined largely by an upper material sheet 708 and a lower material sheet 710. As seen in FIG. 8A, a single strip 706 is disposed within the sealed volume 702. In one embodiment, the material sheets 708 and 710 may include sheets of thin foil that have been sealed together to form the individual volumes 702. In other embodiments, the material sheets 708 and 710 may include a plastic or a polymer material. In other embodiments, one material sheet (e.g., 708) may be formed of one material, while the other sheet (e.g., 710) may be formed of a different material.

In accordance with another exemplary embodiment, as seen in FIG. 8B, a first hair care composition strip 706A and a second hair care composition strip 706B may be disposed in a single sealed volume 702 and separated by a divider 712 (e.g., another material sheet of packaging material). The first strip 706A may include a particular hair care ingredient (e.g., shampoo) while the second strip 706B may include a second hair ingredient (e.g., conditioner). In such an embodiment, a single volume 702 may include the necessary ingredients for a single dose of a desired hair treatment. As such, individual doses of a given hair care treatment may be maintained separate from each other in order to preserve the hydration status of each individual dose. As indicated above, such a hair care treatment may be an application of a single hair care ingredient (e.g., only shampoo, only conditioner, or only, coloring) or it may include multiple ingredients such as the shampoo and conditioner combination shown in FIG. 8A. Additionally, a single strip 706 (or capsule or other discrete physical component) may include multiple hair care ingredients as has been previously discussed.

It is also noted that other forms of the hair care composition such as described above (e.g., microcapsules) may be similarly packaged in individual volumes. Additionally, other packaging structures or devices may be utilized while still maintaining separation between individual doses of the hair care composition (e.g., strips or capsules). For example, discrete containers with removable lids may be utilized containing only a single dose of a hair care treatment. Additionally, while the packaging 700 shown in FIG. 7 shows multiple volumes 702 in a single package, such volumes 702 may be singulated so that individual doses may transported independently.

The preceding description has been presented only to illustrate and describe the present method and products. It is not intended to be exhaustive or to limit the disclosure to any precise form disclosed. Many modifications and variations are possible in light of the above teaching. It is intended that the scope of the present system and method be defined by the following claims.

What is claimed is:

1. A hair care composition package, comprising:
a plurality of sealed volumes; and
a single dose of a desired hair care treatment in each sealed volume, each discrete hair care treatment comprising:
a water swellable component; and
a hair care ingredient associated with the water swellable component.

2. The package of claim 1, wherein said water swellable component comprises a hydrophilic polymer.

3. The package of claim 1, wherein said hair care ingredient comprises one of a shampoo or a conditioner.

4. The package of claim 1, wherein the single dose of desired hair care treatment is formed as a single thin strip of material.

5. The package of claim 1, wherein the single dose of desired hair care treatment includes a first thin strip of material comprising a first hair care ingredient and a second thin strip of material comprising a second hair care ingredient.

6. The package of claim 5, wherein the first hair care ingredient includes a shampoo and wherein the second hair care ingredient includes a conditioner.

7. The package of claim 5, wherein each sealed volume further comprises a divider disposed between the first thin strip of material and the second thin strip of material

8. The composition of claim 1, wherein said hair care ingredient comprises one of an anti curl lotion, an anti humectant pomade, a color glaze, a color mousse, a corrective styling mousse, a cover gray, a curl defining gel, a defining cream, a detangler, an ethnic relaxer, a foam mask, a foaming pomade, a foaming styler, a gel, a hair gloss, a straightener, a keratin treatment, a tonic, a molding cream, a non-permanent hair color, a pre-shampoo treatment, a protecting spray, a regrowth treatment, a relaxing serum, a restructuring serum, a root lift, a root pump, a sculpting gel, a shine pomade, a silk therapy, a smoothing cream, a smoothing mask, a smoothing serum, a straightening balm, a strengthener, a thickening lotion, a vitamin, a volume booster, or a volumizing gel.

9. The composition of claim 1, wherein said water swellable component comprises one of an acrylic polymer, a poly alkylene oxide, a cross-linked polyethylene oxide co-polyurethane hydrogel, a polyvinyl alcohol, an ethylene maleic anhydride copolymer, a polyvinylether, a polyacrylic acid, a polyvinylpyrrolidone, a polyvinylmorpholine, a polyamine, a polyethyleneimine, a polyquaternary ammonium, a saponified copolymer of vinyl acetate-acrylic acid ester, a hydrolyzed copolymer of acrylonitrile or acrylamide, a partially-neutralized crosslinked poly(acrylic acid), a natural based polysaccharide polymer, or a synthetic polypeptide.

10. The composition of claim 1, wherein said hair care ingredient is microencapsulated in said water swellable component.

11. The composition of claim 10, wherein said microencapsulated hair care ingredient is joined to other microencapsulated hair care ingredients by a hydrophilic binder to form a substrate.

12. The composition of claim 1, wherein said hair care composition comprises between approximately 80.00% to about 99.9% hair care ingredient.

13. The composition of claim 1, wherein each hair care treatment further comprises a scented polymer.

14. A method of forming and packaging a hair care composition, the method comprising:
providing a solution having a water swellable component and a first hair care ingredient;
forming a thin, substantially uniform layer of material from the solution;
drying the layer of material;
shaping the dried layer of material into a first plurality of strips; and
packaging each individual strip of the first plurality in its own discrete sealed volume for subsequent access by a user.

15. The method according to claim 14, wherein forming a thin, substantially uniform layer of material from the solution includes spinning the solution in a centrifugal drum.

16. The method according to claim 14, wherein packaging each individual strip in its own discrete sealed volume includes forming each discrete volume using a biodegradable material.

17. The method according to claim 14, wherein the first hair care ingredient comprises a shampoo or a conditioner.

18. The method according to claim 14, further comprising:
providing a second solution having a water swellable component and a second hair care ingredient;
forming a second thin, substantially uniform layer of material from the second solution;
drying the second layer of material;
shaping the dried second layer of material into a second plurality of strips; and
packaging each individual strip of the second plurality one of the discrete sealed volumes along with an associated one of the first plurality of individual strips.

19. The method of claim 18, further comprising placing a divider in each discrete volume between each first strip and its associated second strip.

20. A method of packaging a hair care composition, the method comprising:
providing a hair care composition having swellable component and a hair care ingredient;
forming individual doses of the hair care composition; and
packaging each individual dose in its own discrete sealed volume for subsequent access by a user.
